(19) United States
(12) Patent Application Publication
(10) Pub. No.: US 2013/0012151 A1
(43) Pub. Date: Jan. 10, 2013

(54) Defibrillator with integrated telecommunications

(76) Inventor: Mark S. Hankins, Land O Lakes, FL (US)

(21) Appl. No.: 13/135,368

(22) Filed: Jul. 5, 2011

(51) Int. Cl. H04W 4/22; A61N 1/08; A61N 1/39

(52) U.S. Cl. 455/404.1; 607/5

(57) ABSTRACT

A portable cardiac defibrillator for resuscitating individuals suffering cardiac events having an integrated short-range wireless transmitter or transceiver capable of using its access to another communications device for purpose summoning emergency aid (utilizing the cellular telephone system, satellite telephone system and/or marine or other radio frequencies), and/or providing voice communications with individuals who can provide aid, including in some embodiments allowing for the user to manually provide GPS coordinates in situations where cellular locator service is lacking. Additionally, the combined unit may provide a means for utilizing its communication facilities independent of the defibrillator component, and may utilize such facilities to provide diagnostics of the device to prompt for maintenance when necessary, and to alter or upgrade the device's functionality, including such alteration during its use in an emergency.

### FIELD OF THE INVENTION

This invention relates to addition of short-range wireless communications capability to a defibrillator, and is particularly concerned with a portable automated external defibrillator (AED) which is easily transported and may be used in the field independently of main electrical supply, and which is designed to be used by those relatively untrained persons supplying first aid, rather than by trained first responders.

### BACKGROUND

In the medical field defibrillators are used for the correction of cardiac arrest due to ventricular fibrillation. All such events will require the intervention of first responders in addition to a person or persons providing first aid.

Present automated external defibrillators lack integrated wireless voice communications. The claimed invention consists of a portable defibrillator with a Bluetooth, wi-fi or other interface protocol enabling communication to and from an integrated telephone or other transmitter or transceiver. Such communication is desirable because the defibrillator may be activated for purposes of saving the victim of a serious cardiac event in circumstances where land line telephone service is unavailable, or the party(ies) attempting to resuscitate the victim are too occupied with the resuscitation efforts to use them during the first moments of said efforts. Failure to contact emergency personnel as soon as practicable during the resuscitation effort is associated with poorer patient outcome, in extreme cases including permanent disability and even death. Further, once an interface to said communication service (or services) is integrated into the defibrillator, it makes sense to make it usable without having to use the defibrillator, since not all emergencies requiring such communications require shocks or are even cardiac events. Because the defibrillators are designed for use by untrained personnel, a unit brought to the patient may ultimately not be needed for purposes of defibrillation, however there will very likely be a need to provide care to the victim before paramedics arrive, and conversely there may also be a need for ill-advised attempts at care to be averted by trained personnel providing coaching over the applicable communications system. AEDs routinely provide a certain amount of instruction to the user, which may include text and voice prompts, and in some cases even a full-motion video screen. None of these can provide an adequate substitute for a trained telephone operator (either at a 911 center or a third-party security service) who has access to a computer database with branching scripts that allow for flexible responses to the user as situations arise, and who potentially has the ability to alter the operation of the AED or even change the spoken language or dialect in which it issues its verbal or written prompts.

It is important to note that most AEDs on the market are routinely housed in wall cabinets which bear the legend “Call 911” and either have “Call 911” embroidered on their carrying cases or emblazoned on the units themselves, or which tell the user to do so by voice prompts and/or visual instructions on the display. The need for emergency services to be summoned is strongly recognized; what has not heretofore been recognized and provided for is that in an unpredictable lifesaving situation, providing the instruction to do so without closing the circle as much as possible with respect to the means to carry it out does not provide the gold standard of care and may even be counterproductive, disheartening a would-be rescuer or distracting them from caring for the victim.

While some systems such as the one disclosed in the McSheffrey U.S. Pat. No. 7,961,089 are designed to monitor stations where defibrillators, fire extinguishers and the like are kept, it seems more logical to monitor the actual device because the monitoring system could also provide communications between the user and emergency personnel, and also because in certain circumstances the defibrillator might be used a considerable distance from the station where it was normally kept, and such station monitoring is at risk of misdirecting first responders, who would then lose precious time in being required to determine where the device in question (and the patient) could be located.

As an example, such device might have been brought to a different floor of the building where the victim is located, or might have been brought from the administration building of a high school to its football field. In either case, first responders might find themselves arriving at the station where the device was kept, but unless someone was left behind there to guide them, they might not be able to immediately ascertain the location of the patient, and in some extreme scenarios they might not timely find the patient.

The invention disclosed in the Medema U.S. Pat. No. 7,289,029 provides a different solution to the lack of telecommunication by integrating a cellular phone or other communication device with the AED. While said method does provide some advantages (namely 100% certainty that the cellular phone communication device is present), it has not reached the market in the four years since it issued. Many factors may have prevented its timely adoption, but one of the most daunting factors is obsolescence. Cellular phone technology moves at a gallop, and any phone integrated into the AED device may be obsolete and perhaps even thereby unusable within a few years. For instance in February 2008, analog cellular service was turned off throughout the United States. While few if any cellular phone users were affected, owners of General Motors vehicles equipped with OnStar lost service if their system was among the first four generations of OnStar equipment. Bluetooth (and/or wi-fi or the like) technology will sap less power from what is already a power-intensive lifesaving process, and is simpler, cheaper, more stable and less likely to become obsolete.

Every business or other facility has one or more responsible parties who could pair their own (or company-issued) mobile device(s) with the AED in order to provide it with a potential link to the telephone grid well in advance of any emergency, and/or said AED could be made a node on a wireless Local Area Network (LAN) having Voice Over Internet Protocol (VOIP) access to the telephone grid. In the event of an actual emergency, the AED would initiate a speakerphone call either at the responsible person's command or even on its own at a point in time well before the responsible person self-initiated such a call.

In terms of phone/defibrillator pairings, nonpatent prior art also exists in the form of the AED cabinet and cell phone combination sold by AA Communications of Noblesville, Ind. (http://www.911phone.net/aed.htm). The company's cabinets come equipped with a refurbished Motorola cell phone. While the inclusion of a cellular phone is commendable and the cabinets are tamper resistant, there is provided no means to be assured that the phone is even present, much less charged when an emergency arises. Further, the phone's operation may not be familiar to users of other types of phones, and indeed the company has attempted to remedy this lack by addition of an instruction sticker on the back of the phone. The phone is not attached to the AED or carried in its carrying case, and could be dropped by a person rushing to give first aid to a victim, and conceivably the aid giver would realize the phone had been dropped only after reaching the victim, with the loss of precious minutes or seconds to retrieve the phone, which might have been damaged by the impact with the ground or become lost in the meantime during extreme weather or other some other tumultuous situation. There is also no communication between the AED and the provided phone. The phone depicted in their advertising is also several generations out of date, and may lack location services and GPS. Further, the phone is not contracted to any cellular provider and relies for its connectivity solely on the legal requirement that all cellular providers allow 911 calls to connect from any handset free of charge. Said legal requirement could be altered or removed by subsequent legislative or regulatory action. The phone also depends on the sound decisionmaking of the first aid provider to assure its proper use, or that it is used at all. Properly integrating the communications into the defibrillator by Bluetooth or wi-fi will remove most if not all of these detriments as well as providing the manufacturer and users with assurance that communications will be available, easily operated and appropriate to the environment where the defibrillator is envisioned to be placed.

Likewise, application 20110130665 Method And System For Expediting The Rescue Of Victims Experiencing Sudden Cardiac Arrest (SCA) When Used In Conjunction With An Automated External Defibrillator (AED) pursues a less-than-optimal strategy of providing a device that is not an AED, but which attempts to locate one when it is deemed necessary according to programming, and which also attempts to prioritize the order in which AEDs are provided to patients on a triage basis in a mass-casualty event. Most events potentially requiring use of an AED will not be mass casualty events, and it makes more sense to simply have an AED known to be within a reasonable distance of the patient than to use a device to attempt to locate an AED. The purpose of the invention described in the application differs from the present invention in that it is not intended to assist in communications with call centers or to summon first responders.

Further, application 20110060378 Automated External Defibrillator Device With Integrated Wireless Modem provides a half-solution with only patient data being transmitted. An example of a defibrillator which provides this capability is the LIFEPAK 15 by Physio-Control, Inc. Although said unit has an AED mode, it is intended for professional, day-to-day non-automatic use and the Bluetooth capabilities included are intended to allow it to interface with other medical devices and portable computers, not the telephone grid. Although the professional users of defibrillators need voice communications, in almost all cases they will already have dedicated voice communications to appropriate remote parties. As described, the Lifepak product's communications also seem similar to application 20040127774 entitled Communicating Medical Event Information.

The provision of communications capabilities to respond as emergencies arise has long been recognized as a requirement of due care in American jurisprudence. First year law students are invariably taught the landmark 1932 T.J. Hooper case in their torts classes. In that case, a tugboat whose barge sank was found unseaworthy (and thus the owner of its towed cargo had to be compensated by the tugboat owner) because the vessel lacked a radio with which to learn of approaching bad weather, even though provision of radios on vessels of its type was not then customary. In short, where communications could be provided to mitigate an emergency situation, famed judge Learned Hand ruled that it was negligent to fail to provide it, stating: “There are precautions so imperative that even their universal disregard will not excuse their omission.” Thus, the present invention responds to an undeniable unmet need with respect to most automatic external defibrillators: because contacting first responders will invariably be required when the defibrillator is used, it would be negligent to omit such communication capability when the easy availability (and usability) of such communications without distracting the first aid provider (or the mitigating presence of trained first responders) cannot be assumed. Only defibrillators located in such places as fire stations and hospitals could conceivably be exempt from the requirement for integrated communications. Since no defibrillators having call-out capability have reached the market in the near decade since one was proposed, a departure from the initial approach is called for.

Additionally, it is undeniable that the costs for the basic technology that makes up a Bluetooth or wi-fi link to a cellular phone or wireless LAN have declined dramatically since their introduction, to the point where embedding them in various devices which may see infrequent use is no longer cost-prohibitive, nor does it unreasonably accelerate the obsolescence of said devices.

Further, the ability to report the condition of a building from a remote location to a central monitoring hub has long been a fixture of the fire alarm industry, and has in recent years been adapted for automotive uses by services such as OnStar and by vending machine manufacturers. A piece of lifesaving equipment such as an AED must not be allowed to be found in an unready condition when an emergency arises, and self-diagnostics and self-reporting to a remote central monitoring hub are the most desirable way of assuring such readiness. To provide such mechanisms to assure the availability of soft drinks but not to optimize the readiness of lifesaving equipment would be extremely callous.

In the preferred embodiment of the invention, said Bluetooth link to a wireless telephone immediately contacts a service, such as the existing service known as OnStar, and the call is routed to agents specifically trained to coach callers in the course of using the manufacturer's portable defibrillators to respond to serious cardiac events, and said agents also will be responsible for summoning emergency personnel to the scene using location information provided by the telephone component of the invention or by its user.

In an alternative embodiment of the invention, said Bluetooth connection to a wireless telephone is not subscribed to any security provider's service, but calls 911 automatically when the defibrillator is activated.

In another alternative embodiment of the invention, the defibrillator utilizes the conference call capability of the host phone to simultaneously initiate calls both to a service and to 911.

In another alternative embodiment of the invention, said defibrillator is packaged with a satellite telephone transceiver intended for use in areas where cellular service is unavailable, and it calls a predetermined phone number and said call is routed to agents trained specifically to coach callers in the course of using portable defibrillators to respond to serious cardiac events, and said agents also will be responsible for summoning emergency personnel to the scene using location information provided by the telephone.

In another alternative embodiment of the invention, said defibrillator is packaged with a radio transceiver intended for use in areas where cellular service is unavailable, and it uses a recognized distress frequency in the marine or other radio band(s).

In another alternative embodiment of the invention, said defibrillator also includes a Global Positioning System display so that the person providing aid can also provide coordinates to third parties responding to a radio call on a recognized distress frequency.

In another alternative embodiment of the invention, said defibrillator also includes a Global Positioning System with a voice synthesizer so that the person providing aid can also provide coordinates to third parties responding to a radio call on a recognized distress frequency by means of activating said voice synthesizer rather than speaking the coordinates.

In another alternative embodiment of the invention, said defibrillator is packaged with an EPIRB distress beacon, which may or may not be designed to activate automatically when the defibrillator is in use.

In another alternative embodiment of the invention, said defibrillator does not automatically summon aid, rather it prompts the user to summon aid based on comparing preprogrammed criteria with information gleaned about the victim's condition. The inclusion of such a prompting system is intended to avoid automatically summoning emergency aid in circumstances where the victim is not suffering an actual cardiac event.

In another alternative embodiment of the invention, said combined unit provides the user with the ability to utilize the communications facilities of the unit without activating the defibrillator portion of the unit.

In another alternative embodiment of the invention, the built-in communications capabilities include data and are leveraged by utilizing them to additionally provide diagnostics regarding the maintenance status of the defibrillator, including but not limited to the state of charge and condition of the battery, as well as the capacitor(s) and pads and also to report that the defibrillator is in fact in place at its assigned station.

In another alternative embodiment of the invention, the built in communications capabilities include data and are leveraged to allow authorized updates to the firmware and software of the defibrillator for purposes of altering or improving its function or, say, the quality of its voice prompts, as an example adding foreign languages or rewording a prompt whose language has proven confusing or otherwise problematic. Such changes may be provided during the emergency, for instance to adapt to a first aid giver who does not speak the same language that has been previously provided to the AED.

In other aspects, the invention provides a system and a computer program having features and advantages corresponding to those discussed above.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific examples of the embodiments disclosed and that modifications and other embodiments are intended to be included within the scope of the appended claims. Although specific terms are employed herein, they are used in a generic and descriptive sense only and not for purposes of limitation.

### REFERENCES

| | | |
|---|---|---|
| 1,936,405 | November 1933 | Mueller |
| 3,258,013 | June 1966 | Druz |
| 4,023,573 | May 1977 | Pantridge et al |
| 4,610,254 | September 1986 | Morgan et al |
| 4,904,983 | February 1990 | Mitchell |
| 4,974,607 | December 1990 | Miwa |
| 5,081,667 | January 1992 | Drori et al |
| 5,619,991 | April 1997 | Sloane |
| 5,666,404 | September 1997 | Ciccotelli et al. |
| 5,701,904 | December 1997 | Simmons et al. |
| 5,704,364 | January 1998 | Saltzstein et al. |
| 6,181,981 | January 2001 | Varga, et al |
| 6,388,617 | May 2002 | Flood et al |
| 7,289,029 | October 2007 | Medema et al |
| 7,860,725 | December 2010 | Gopinathan et al. |
| 7,961,089 | June 2011 | McSheffrey et al. |

Murakami H. et al. “Telemedicine Using Mobile Satellite Communication” IEEE Transactions on Biomedical Engineering, IEEE Service Center, Piscataway, N.J., vol. 41, No. 5, May 1994, pp. 488-497, XP000770263 ISSN: 0018-9294

AA Communications of Noblesville, Ind. (http://www.911phone.net/aed.htm).

Meet the Smart Coke Machine; The Sacramento Bee Business Technology; Wednesday, Aug. 30, 1995.

Skywire Provides Details of Wireless ‘VendView’ System; Vending Times, September, 1994.

Skywire allows vendor tracking of pop stock and sales details; RCR, vol. 14, No. 17, Sep. 4, 1995.

Left high and dry? Sold-out machine sends for Cokes; Nashville Banner, Wednesday, Aug. 16, 1995.

Coke machines signal when it's time for a refill; The Globe & Mail, Toronto, ON, Aug. 30, 1995.

Wireless Communications Forum; vol. III, No. 1, Apr., 1995, pp. 25-30.

In re Eastern Transportation Co. (The T.J. Hooper), 60 F.2d 737 (2d Cir. 1932)

Rainer and Cegielski, Introduction to Information Systems: Enabling and Transforming Business, John Wiley and Sons, 2009, ISBN 0470473525, 9780470473528 p. 261 (authors state that MedAire remotely controls defibrillators, however Medaire's website offers the HeartStart FRx, which is not remotely controlled).

### BRIEF DESCRIPTION OF THE DRAWINGS

Having thus described the invention in general terms, reference will now be made to the accompanying drawings, which are not necessarily drawn to scale, and wherein:

FIG. 1 shows schematically a block diagram of a defibrillator with communication-equipped AED according to one embodiment of the present invention.

FIG. 2 is a flow chart illustrating a number of steps of a method according to one embodiment of the present invention.

FIGS. 3a-c illustrate the appearance of user interface elements on a display of a AED during operation of the method of FIG. 2.

### DETAILED DESCRIPTION OF THE INVENTION

The present inventions now will be described more fully hereinafter with reference to the accompanying drawings, in which some examples of the embodiments of the inventions are shown. Indeed, these inventions may be embodied in many different forms and should not be construed as limited to the embodiments set forth herein; rather, these embodiments are provided by way of example so that this disclosure will satisfy applicable legal requirements. Like numbers refer to like elements throughout.

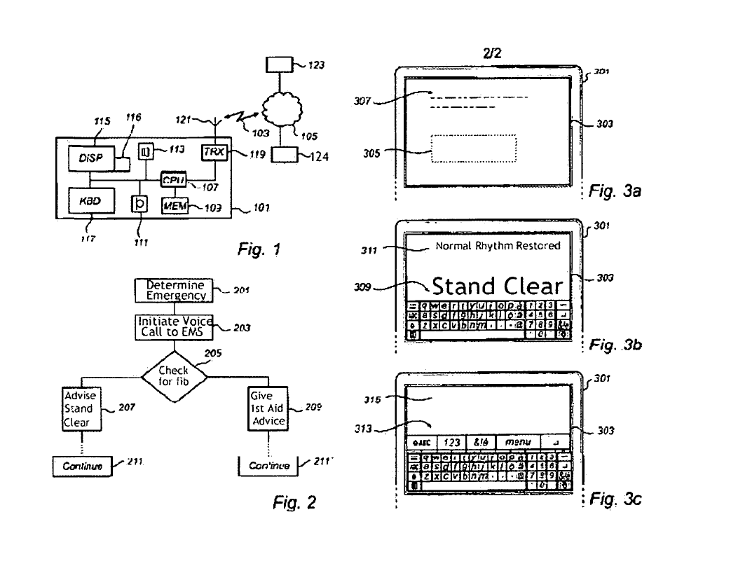

FIG. 1 illustrates schematically a communication-equipped AED 101 in which an embodiment of the present invention is implemented. The AED 101 is capable of communication via a Bluetooth or wi-fi air interface 103 with a radio communication system 105 such as the well known systems GSM/GPRS, UMTS, CDMA 2000, etc. The AED comprises a processor 107, memory 109 as well as input/output units in the form of a microphone 111, a speaker 113, a touch sensitive display 115. The touch sensitive display 115 comprises appropriate touch sensing means, such as electronic sensing circuitry 116, configured to sense touch by way of a finger tip.

The communication-equipped AED 101 may for example incorporate Bluetooth circuitry or be equipped with a similar communication means. The method according to the present invention will in general reside in the form of software instructions, together with other software components necessary for the operation of the AED 101, in the memory 109 of the AED. Any type of electronic memory is possible, such as a hard drive, a semi-permanent storage chip such as a flash memory card or “memory stick” etc. The software instructions of the inventive notification function may be provided into the memory 109 in a number of ways, including distribution via the network 105 from a software supplier 123. That is, the program code of the invention may also be considered as a form of transmitted signal, such as a stream of data communicated via the Internet or any other type of communication network, including cellular radio communication networks of any kind, such as GSM/GPRS, UMTS, CDMA 2000 etc. Likewise, the voice communications provided by the device occur with a call center 124.

Turning now to FIGS. 2 and 3a-c, a method according to one embodiment of the invention will be described in terms of a number of steps to be taken by controlling software in an AED such as the AED 101 described above in connection with FIG. 1.

The exemplifying method starts at a point in time when the AED is self-activated on a predetermined schedule, at which point it reports its status and requests and obtains any software or firmware updates before shutting down again, or activated by a user, causing user interface elements in the form of initial instructions to be displayed on a touch sensitive display 303 of an AED 301, as well as voice prompts to issue from its speaker. As the skilled person will realize, any amount of displayed information may also be present on the display 303 as indicated by schematically illustrated dummy content 307.

A touch action to indicate an emergency, e.g. tapping, performed by a user on the screen 303 is sensed in a sensing. step 201. The sensing is realized, as discussed above, in a touch sensing means, such as sensing circuitry connected to the display 301 (cf. sensing circuitry 116 in FIG. 1).

In a call-out step 203, the AED that has been notified by the user there is an actual emergency immediately summons aid. The determination of the need for aid is made by a user selection through touch or voice response and the steps necessary to summon said aid are generally implemented by computer instructions stored in a memory device, such as memory 109, and executed by processor 107.

In a resuscitation step 205, the input received from the pads is used to select between two alternatives (shock needed, or no shock needed) for presenting subsequent user interface elements on the display 303. Like the determining means, the selection of resuscitation or not and the manner of presentation of the user interface elements is typically performed by control means that is generally implemented by computer instructions stored in a memory device, such as memory 109, and executed by processor 107.

In a case where the need for a shock is determined, the user is advised to get clear of the patient step 207. This is illustrated in FIG. 3b where user interface elements in the form of an instruction 309 is displayed directing the user to stand clear. A text output field 311 is also indicated in which any subsequent instructions to the user (e.g., the need to repeat the shock) is to be displayed during a continuation as indicated by reference numeral 211. Said field could also report the user's GPS coordinates so the user could speak them to the emergency call center in the event they were not automatically transmitted.

In a case where a shock is not indicated, the, a user interface giving general first aid instructions is displayed in a display step 209. This is illustrated in FIG. 3c where user interface elements in the form of text or graphics 313 are displayed. A text output field 315 is also indicated in which any subsequent user input (e.g. results due to tapping on a displayed keyboard 313 in response to prompts) is to be displayed during a continuation as indicated by reference numeral 211.

Although the example above only shows user interface elements in the form of keyboard keys, other elements are also possible, such as user interface elements in the forms of scroll bars, editing windows, dialog boxes etc. Moreover, a plurality of elements may be grouped together and configured such that, e.g. in a case with input keys, one single displayed key is associated with the group of keys.

In addition to or instead of displaying the user interface elements, the AED can provide voice prompts. For example, the user interface can display user interface elements while simultaneously speaking them to reinforce their importance.

As the user is operating the AED, simultaneous with its operation the communication element of the AED is functioning so that the voice of one or more remote operator(s) is heard over the speaker, and the microphone allows the user to speak to the operator(s). In addition, a remote operator may be permitted to alter aspects of the AED's operation (e.g., changing the prompts on the screen to a foreign language) as necessary. Such changes are typically performed by control means that is generally implemented by computer instructions stored in a memory device, such as memory 109, and executed by processor 107.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific examples of the embodiments disclosed and that modifications and other embodiments are intended to be included within the scope of the appended claims. Although specific terms are employed herein, they are used in a generic and descriptive sense only and not for purposes of limitation.

The embodiments of the invention in which an exclusive property or privilege is claimed are defined as follows:

1. A portable defibrillator with an integrated wireless communication system which is automatically activated when the defibrillator is put into use, or at other times when said activation may be appropriate.

2. The system of claim 1 with a voice and/or data communication system having indirect access to the telephone grid through Bluetooth, wi-fi or similar pairing with one or more mobile phones, which may be satellite phone(s).

3. The system of claim 1 with a voice and/or data communication system having indirect access to recognized marine or other emergency voice communication frequencies through Bluetooth, wi-fi or similar pairing with one or more transceivers.

4. The system of claim 1 with an integrated Global Positioning System display.

5. The system of claim 1 with an integrated Global Positioning System having an integrated voice synthesizer for transmittal of coordinates over the voice communication system.

6. The system of claim 2 with an integrated wireless voice and/or data communication system having access to the telephone grid that is arranged such that activation of the defibrillator immediately causes the voice communication system to contact a predetermined telephone number.

7. The system of claim 6 where operators trained in responding to cardiac events are standing by to coach the rescuer and summon emergency personnel.

8. The system of claim 6 where the predetermined telephone number is 911 or a similar public emergency phone number.

9. The system of claim 2 where the voice and/or data communication system has access to the telephone grid through satellite telephony that is arranged such that activation of the defibrillator immediately causes the voice communication system to contact a predetermined telephone number.

10. The system of claim 9 where the predetermined telephone number reaches operators trained in responding to cardiac events who are standing by to coach the rescuer and summon emergency personnel.

11. The system of claim 6 where the defibrillator is capable of initiating two telephone calls simultaneously using a host phone's conference feature, one to local 911 as in claim 8 and the other to a predetermined telephone number with operators trained in responding to cardiac events as in claim 10.

12. The system of claim 2 which also provides data telemetry regarding the individual suffering the cardiac event.

13. The system of claim 1 which may be activated without utilizing the defibrillator portion of the integrated unit.

14. The system of claim 1 in which the communications system is not automatically activated when the defibrillator is put to use.

15. The system of claim 1 where the defibrillator is programmed with a voice prompt that indicates whether the user should summon emergency aid based on criteria obtained by the system's monitoring of the patient.

16. The system of claim 1 where the defibrillator is provided with the capability to send data regarding its location and operational readiness and/or need for maintenance to a monitoring location.

17. The system of claim 1 where the defibrillator is provided with the capability to receive data from a remote location such as software and firmware updates to improve its functionality.

18. The system of claim 1 where the wireless communication portion of the defibrillator is housed and connected in a modular fashion such that it may be removed and replaced with a component of the same type or of a different type.
